(19) United States
(12) Patent Application Publication
(10) Pub. No.: US 2019/0380843 A1
(43) Pub. Date: Dec. 19, 2019

(54) APPARATUS FOR INSERTING CAGE BETWEEN VERTEBRAE

(71) Applicant: SOLCO BIOMEDICAL CO., LTD., Pyeongtaek-si (KR)

(72) Inventors: Seo Kon Kim, Anyang-si (KR); Il Kim, Seoul (KR); Myung Heon Ha, Osan-si (KR)

(21) Appl. No.: 15/542,960

(22) PCT Filed: Feb. 23, 2017
(86) PCT No.: PCT/KR2017/001982

§ 371 Date: Jul. 12, 2017

(30) Foreign Application Priority Data
Jan. 31, 2017 (KR) 10-2017-0014055

(51) Int. Cl. A61F 2/46; A61B 17/34; A61F 2/44

(52) U.S. Cl. CPC A61F 2/4611; A61B 17/7071; A61F 2/4455; A61B 17/3468

(57) ABSTRACT

The invention relates to an apparatus for inserting a cage between vertebrae, according to the inventive concept, includes a body accommodating an inner rod coupled to the cage, which is to be inserted between vertebrae, a push bar installed in the body to be slid and configured to push one side of the cage as the push bar is moved forwards from the body, a support bar supporting an opposite side of the cage and installed in the body to be slid to move rearwards when the push bar is moved forwards, and an adjustment unit installed in the body to be manipulated and configured to adjust movements of the push bar and the support bar such that a rotation amount of the cage is adjusted according to manipulation by a user.

### BACKGROUND

The inventive concept relates to an apparatus for inserting a cage between vertebrae, and more particularly, relates to an apparatus for inserting a cage between vertebrae, which may stably and simply insert a cage between vertebrae.

An intervertebral disc is a circular cartilage disc inserted between vertebrae, and performs a shock absorbing function of absorbing a weight of and a shock on a body at a location between vertebrae except for some of cervical vertebrae and dispersing the shock, which is like a spring. In this case, the intervertebral disc functions to hold the vertebrae such that the vertebrae are not dislocated, make the range of an intervertebral foramen smooth by separating two vertebrae such that a vertebral nerve is not compressed, and make movement of each vertebra smooth.

Such an intervertebral disc includes an annulus fibrosus and a nucleus pulposus. The annulus fibrosus adjusts movement of pieces of the vertebrae, and the nucleus pulposus therein includes 70-80% of water content. The intervertebral disc absorbs or transfers a weight and a shock that are vertically applied. In degenerative disc diseases, movement of the annulus fibrosus or capability for the annulus fibrosus to contain the nucleus pulposus is weakened, and the water content of the annulus fibrosus is reduced. Due to such a complex result, diseases such as spinal stenosis, forming of osteophyte, disc prolapse and compression of a nerve root.

An example of a method for treating such diseases accompanied by the intervertebral disc includes a method for replacing a space between two adjacent vertebral bodies with an artificial disc or an implant, for example, a so-called cage after a damaged intervertebral disc of a human body is removed. That is, as the cage is implanted, a natural state is made as much as possible. An original distance between the two adjacent vertebral bodies, which is an original height of the intervertebral disc, is restored, so that a function of the vertebrae is restored.

In recent years, transforaminal lumbar interbody fusion (TLIF) is proposed as a surgery method for inserting such a cage between vertebrae. The TLIF, which is one of vertebral body fusions, is a surgery method for approaching a rear side of the human body and inserting a cage. In detail, the TLIF is a surgery of inserting a cage using an insertion device while removing vertebral joints in a direction in which a neuropore protrudes after a surgical site is incised along opposite sides of a spinal muscle and is minimally exposed such that a screw may be fixed. The TLIF has advantages in that an amount of bleeding is small and an operation time may be shortened.

In the TLIF, for minimal incision and minimization of interference in a human body, generally, after a tip end of the cage is firstly inserted through a rear surface (a side of a back) of the human body and is located between the vertebrae, a side surface of the cage is arranged toward a front surface (a side of an abdomen of the human body) of a portion between the vertebrae, and thus, the insertion of the cage is completed. That is, an impactor which is an auxiliary tool is required such that the side surface of the inserted cage faces the front surface of the portion between the vertebrae. Force is applied to the side surface of the cage using the impactor to rotate the cage, so that the cage may be arranged. However, a surgery using the impactor has a disadvantage in that the surgery is difficult or success in the surgery cannot help depending on an operational ability of an operator.

Technologies disclosed in Korean Patent No. 10-1273199 (hereinafter, referred to as “Patent document 1”) and Korean Patent No. 10-0371308 (hereinafter, referred to as “Patent document 2”) are proposed as a method for facilitating location adjustment of the cage. An apparatus for inserting a cage according to the prior arts is configured to implement an articulated rotary motion of pivoting a cage after inserting the cage such that a tip end of the cage faces a front surface of a portion between vertebrae, which is a so-called articulating mechanism.

In the apparatus for inserting a cage in which such an articulating mechanism is implemented, it is very important that the cage keeps a standing posture without rotation while the cage is inserted and a posture of the cage is precisely adjusted while the cage is rotated to a right position after the cage is inserted. Further, various researches and developments are performed to solve the above-described technical problems.

### SUMMARY

The inventive concept is conceived to solve the above-described technical problems, and an aspect of the inventive concept is to provide an apparatus for inserting a cage between vertebrae, which may stably maintain a posture of the cage while the cage is inserted and may precisely adjust the posture of the cage when the cage is rotated.

Technical aspects to be achieved by the inventive concept are not limited to the above-described technical aspects, and not-mentioned other technical aspects should be clearly understood by those skilled in the art to which the inventive concept pertains, based on the following description.

To implement the above-described problems, disclosed is an apparatus for inserting a cage between vertebrae, the apparatus including a body accommodating an inner rod coupled to the cage, which is to be inserted between vertebrae, a push bar installed in the body to be slid and configured to push one side of the cage as the push bar is moved forwards from the body, a support bar supporting an opposite side of the cage and installed in the body to be slid to move rearwards when the push bar is moved forwards, an adjustment unit installed in the body to be manipulated and configured to adjust movements of the push bar and the support bar such that a rotation amount of the cage is adjusted according to manipulation by a user.

According to the apparatus for inserting a cage between vertebrae according to the inventive concept, a curved surface supporting part supporting a curved part of the cage may be formed at a tip end of the support bar.

According to the apparatus for inserting a cage between vertebrae according to the inventive concept, the adjustment unit may include a rotary handle installed in the body to be rotatable.

According to the apparatus for inserting a cage between vertebrae according to the inventive concept, a power transmission unit configured to move the support bar and the push bar in opposite directions as the adjustment unit is rotated may be installed inside the body.

According to the apparatus for inserting a cage between vertebrae according to the inventive concept, the power transmission unit may include: first and second connectors extending from side surfaces of the push bar and the support bar, respectively, and a rotary body, opposite ends of which are connected to the first and second connectors to be rotatable, and formed in the body to be pivoted as the adjustment unit is rotated.

The apparatus for inserting a cage between vertebrae according to the inventive concept may further include a locking unit configured to lock and unlock the support bar at a first position of the body when the cage is initially inserted between vertebrae.

According to the apparatus for inserting a cage between vertebrae according to the inventive concept, the locking unit may include a manipulation lever installed in the support bar to be rotatable, and a first coupling part provided in the body and detachably coupled to the manipulation lever.

According to the apparatus for inserting a cage between vertebrae according to the inventive concept, the locking unit may further include a second coupling part detachably coupled to the manipulation lever to lock and unlock the support bar at a second position of the body when the cage is rotated.

According to the apparatus for inserting a cage between vertebrae according to the inventive concept, signs indicating a rotation state of the cage may be displayed on the body and the support bar. Here, the signs may include at least one of a character, a symbol and a number indicating the rotation state or an angle of the cage.

The apparatus for inserting a cage between vertebrae according to the inventive concept may further include handles coupled to the body, and handle coupling parts configured to detachably couple the handles to the body.

According to an apparatus for inserting a cage between vertebrae according to the inventive concept, each of the handle coupling parts may include insertion slots formed on both sides of a support part formed in the body, and a pair of protrusion inserting parts formed at an end of the corresponding handle to define an insertion space of the support part and inserted into the insertion slots.

According to an apparatus for inserting a cage between vertebrae according to the inventive concept, the support parts and the insertion slots are symmetrically formed at upper and lower portions of the body such that the handles are coupled to the upper and lower portions of the body.

According to an apparatus for inserting a cage between vertebrae according to the inventive concept, the handle coupling part may further include a fixing unit installed in the protrusion inserting parts to be rotatable to lock or unlock the support part.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a perspective view illustrating an apparatus for inserting a cage between vertebrae according to an embodiment of the inventive concept;

FIG. 2 is an exploded perspective view illustrating a state in which some components of the apparatus for inserting a cage between vertebrae, which is illustrated in FIG. 1;

FIG. 3 is a perspective view illustrating a cage to be inserted between vertebrae, which is illustrated in FIG. 1, and an inner rod coupled thereto;

FIG. 4 is a partial perspective view illustrating a state in which the cage to be inserted between vertebrae is rotated;

FIG. 5 is a view illustrating an operation of the apparatus for inserting a cage between vertebrae according to the embodiment of the inventive concept;

FIG. 6 is a view illustrating a process of inserting the cage according to an operation of the apparatus of FIG. 5 for inserting a cage;

FIGS. 7 and 8 are views illustrating a moving position of a support bar and a state of a locking unit according thereto;

FIG. 9 is a view illustrating an installation form of handles attached to the apparatus for inserting a cage between vertebrae according to the inventive concept;

FIGS. 10 and 11 are views illustrating handle coupling parts for coupling of the handles;

FIG. 12 is a perspective view illustrating an apparatus for inserting a cage between vertebrae according to another embodiment of the inventive concept; and

FIG. 13 is a plan view illustrating the apparatus for inserting a cage between vertebrae, which is illustrated in FIG. 12.

### DETAILED DESCRIPTION

Hereinafter, an apparatus for inserting a cage between vertebrae, which is related to the inventive concept, will be described in more detail with reference to the accompanying drawings.

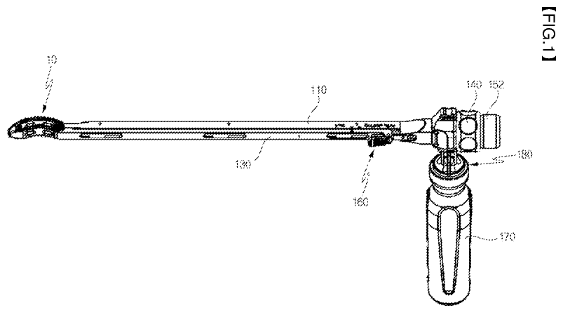

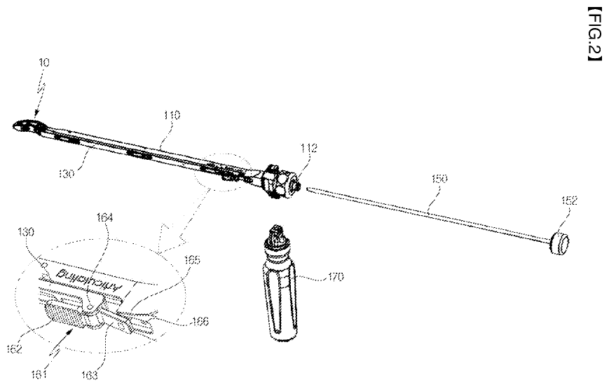

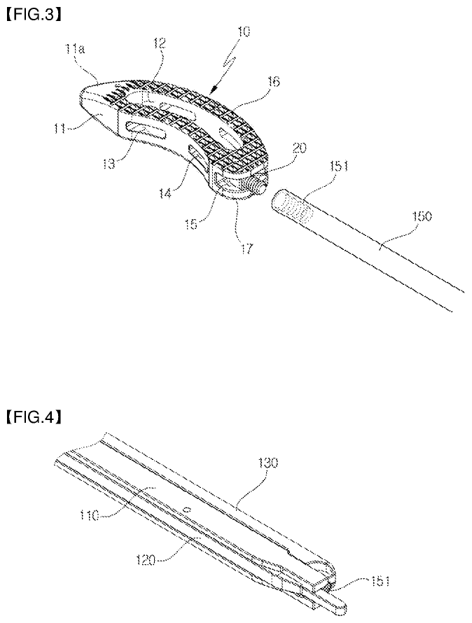

FIG. 1 is a perspective view illustrating an apparatus for inserting a cage between vertebrae according to an embodiment of the inventive concept, FIG. 2 is an exploded perspective view illustrating a state in which some components of the apparatus for inserting a cage between vertebrae, which is illustrated in FIG. 1, and FIG. 3 is a perspective view illustrating a cage to be inserted between vertebrae, which is illustrated in FIG. 1, and an inner rod coupled thereto. Further, FIG. 4 is an exploded perspective view illustrating a state in which the cage to be inserted between vertebrae is rotated, when viewed in a direction that is opposite to that of FIG. 1.

Referring to FIGS. 1 to 4, the apparatus for inserting a cage inserted between vertebrae includes a body 110, a push bar 120, a support bar 130 and an adjustment unit 140.

The body 110 accommodates an inner rod 150 (see FIG. 2) coupled to a cage 10 to be inserted between vertebrae. Referring to FIG. 2, an opening 112 communicating an inside and an outside of the body 110 to each other is provided at a rear end of the body 10, and the inner rod 150 is inserted into the body 110 through the opening 112. A rotation knob 152 for rotating operation is provided at a rear end of the inner rod 150.

Referring to FIG. 3, the cage 10 includes a cage body 11 defining an outer appearance thereof and a rotary member 20 coupled to the cage body 11 to be rotatable

A tip end of the cage body 11 may be inclined and an inclined surface 11a of the tip end may be formed to be vertically symmetric such that insertion may be easily performed. Uneven parts 16 configured to prevent sliding at contact with a vertebral body 1 (see FIG. 6) may be formed on upper and lower surfaces of the cage body 11, and function to make fusion with the vertebral body 1 firmer as well as to prevent the sliding.

A powdered bone accommodating hole 12 in which powdered bones are accommodated may be vertically formed through the cage body 11, and a plurality of side through-holes 13 and 14 allowing the powdered bones to be more smoothly accommodated may be formed on side surfaces of the cage body 11.

The rotary member 20 is connected to a rear end of the cage body 11 to be rotatable. A slot 15 defining a rotation space for the rotary member 20 is provided at the rear end of the cage body 11 and the rotary member 20 is connected to the cage body 11 inside the slot 15 through pin connection or the like to be rotatable.

A male screw thread is formed on an outer peripheral surface of the rotary member 20 and a female screw thread 151 is formed in the inner rod 150 such that the inner rod 150 may be fastened to the rotary member 20. When the inner rod 150 is rotated in one direction in a state in which the inner rod 150 and the rotary member 20 are in contact with each other, the inner rod 150 and the rotary member 20 are screw-fastened to each other, and when the inner rod 150 is rotated in an opposite direction in a state in which the inner rod 150 and the rotary member 20 are fastened to each other, the inner rod 150 is separated from the rotary member 20. In the present embodiment, a case where the male screw thread is formed in the rotary member 20 and the female screw thread 151 is formed in the inner rod 150 has been described as an example. However, the inventive concept is not limited thereto, the opposite case is possible, and any structure is possible as long as the rotary member 20 and the inner rod 150 are fastened to each other in a male-female coupling scheme in the structure.

Referring to FIG. 4, a push bar 120 is installed in the body 110 to be slid. In the present embodiment, a structure in which the push bar 120 is inserted into an inner slide space formed in the body 110 is described as an example. The push bar 120 is moved forwards from the body 110, that is, is moved toward the cage 10, to push one side of the cage 10.

Referring back to FIGS. 1 and 2, a support bar 130 is configured to support the other side of the cage 10. That is, the push bar 120 and the support bar 130 are configured to support two rear portions of the cage 10. Further, the support bar 130 is installed in the body 210 in parallel to the push bar 120 to be slid, and when the push bar 120 is moved forward, is rearward moved in a direction that is opposite to that of the push bar 120, making it possible to implement an articulated rotary motion of the cage 10, which is a so-called articulating mechanism.

The adjustment unit 140 is installed in the body to be manipulated by a user, and adjusts movements of the push bar 120 and the support bar 130 according to manipulation by the user. In this way, a rotational amount of the cage 10, in other words, a rotation angle of the cage body 11 with respect to the rotary member 20 may be adjusted by adjusting the movements of the push bar 120 and the support bar 130 according to the manipulation by the adjustment unit 140.

The adjustment unit 140 may have a shape of a rotary handle that is installed in the body to be rotatable, and in the present embodiment, a state in which the adjustment unit 140 having a shape of a handle is installed at the rear end of the body 110 is described as an example. In this case, the rotation knob 152 of the inner rod 150 is arranged at a rear end of the adjustment unit 140. The movement amounts of the support bar 130 and the push bar 120 may be adjusted according to the rotation amount of the adjustment unit 140. When the adjustment unit 140 is rotated in one direction, the support bar 130 is moved rearward and the push bar 120 is moved forward, and when the adjustment unit 140 is rotated in a direction that is opposite to the one direction, the support bar 130 is moved forward, and the push bar 120 is moved rearward.

To this end, a power transmission unit configured to move the support bar 130 and the push bar 120 in directions opposite to each other according to the rotation of the adjustment unit 140 may be installed inside the body 10. For example, the power transmission unit may include a first power transmission unit configured to transfer a rotational motion of the adjustment unit 140 while converting the rotational motion of the adjustment unit 140 into a linear motion of the support bar 130, and a second power transmission unit configured to transfer the linear motion of the support bar 130 to the push bar 120 in a direction that is opposite to that of the first power transmission unit. Examples of the first transmission unit include a combination of a worm gear and a rack and pinion, and examples of the second power transmission unit include a rack and pinion. However, the first and second power transmission units may be implemented in various modified forms as well as the above-described configuration. Furthermore, the first power transmission unit may convert the rotational motion of the adjustment unit 140 into a linear motion of the push bar 120 and the second power transmission unit may transfer the linear motion of the push bar 120 to the support bar 130. Further, the power transmission unit may not be divided into the first power transmission unit and the second power transmission unit and may be configured to transfer the rotational motion of the adjustment unit 140 to the push bar 120 and the support bar 130, respectively. In addition, the power transmission unit may be implemented using various configurations and mechanisms in addition to the above-described configurations.

Referring back to FIGS. 1 and 2, the apparatus for inserting a cage includes a locking unit 160 configured to lock and unlock the support bar 130 at a first position of the body 110. Here, the first position corresponds to a position into which the cage 10 is initially inserted, that is, a position in which the tip end of the cage 10 faces a front surface of a portion between vertebrae. The locking unit 160 fixes a position of the support bar 130 supporting the cage 10 to prevent the cage from being arbitrarily rotated when the cage 10 is inserted, and accordingly, may provide firm fixing force when the cage 10 is initially inserted.

The locking unit 160 includes a manipulation lever 161 installed in the support bar 130 to be rotatable and a first coupling part 165 provided in the body 110, and the first coupling part 165 may be attached/detached to/from the manipulation lever 161.

The manipulation lever 161 has a structure in which a manipulation part 162 and a fixing part 163 are integrally formed, and may be connected to the rear end of the support bar 130 to be pivoted about a rotary shaft 164. An insertion protrusion may be formed in the fixing part 163, and in this case, the first coupling part 165 may have a shape of an insertion groove in which the insertion protrusion is fitted. The user may pull the manipulation part 162 of the manipulation lever 161 to rotate the manipulation lever 161 so that the fixing part 163 is coupled to the first coupling part 165, and may push the manipulation part 162 to rotate the manipulation lever 161 in an opposite direction so that the fixing part 163 is separated from the first coupling part 165.

The first coupling part 165 may be implemented using various modified structures as long as the structures may be attached/detached to/from the fixing part 163 in a male-female coupling scheme, in addition to the above-described configuration.

Furthermore, the locking unit 160 may further include a second coupling part 166 configured to lock and unlock the support bar 130 at a second position of the body 110 when the cage 10 is rotated. The second position corresponds to a position in which the support bar 130 is spaced rearward apart from the first position by a predetermined distance, and the second coupling part 166 may be detachably coupled to the manipulation lever 161, which is like the first coupling part 165. According to the present embodiment, it is exemplified that the second coupling part 166 has a form of an insertion groove spaced rearward apart from the first coupling part 165 having a form of an insertion groove by a predetermined interval, and the second coupling part 166 may be also implemented in various modified forms, which is like the first coupling part 165. The second coupling part 166 functions to fix a location of the support bar 130 in a state in which the cage 10 is rotated at a predetermined angle, to the fixed state firm.

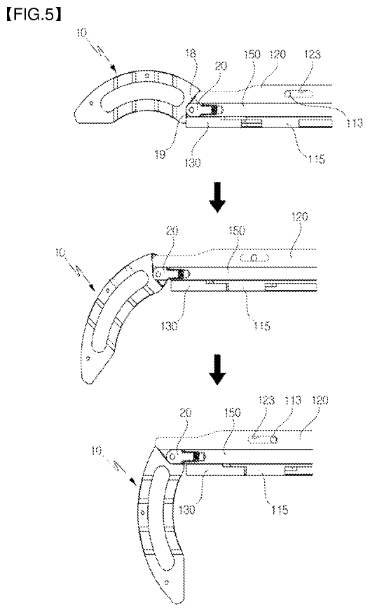

FIG. 5 is a view illustrating an operation of the apparatus for inserting a cage between vertebrae according to the embodiment of the inventive concept, and FIG. 6 is a view illustrating a process of inserting the cage according to the operation of the apparatus of FIG. 5 for inserting a cage.

The user fastens the inner rod 150 and the rotary member 20 of the cage 10 to each other by rotating the rotation knob 152 of the inner rod 150, and fixes the support bar 130 to the first position by operating the locking unit 160. Further, the user moves the apparatus for inserting a cage to an insertion position of the vertebral body 1 to insert the cage 10.

While the cage 10 is initially inserted, the push bar 120 supports a first support wall 18 formed in the slot 15 of the cage body 11, and the support bar 130 supports a second support wall 19 of the cage body 11. Because the location of the support bar 130 is fixed by the locking unit 160, the cage 10 may be stably inserted by firm support force without arbitrary rotation when the cage 10 is initially inserted.

When the locking unit 160 is unlocked after the cage 10 is initially inserted, a state in which the support bar 130 may be freely moved is achieved. In this state, when the adjustment unit 140 is rotated, the push bar 120 is moved forward to push the first support wall 18 of the cage body 11, and the support bar 130 is moved rearward to provide a rotation space of the cage body 11, so that an articulated rotary motion of the cage 10 is achieved. In this process, because the rotation amount of the cage 10 may be adjusted according to the rotation amount of the adjustment unit 140, a posture of the cage 10 may be precisely adjusted during surgery.

When the adjustment unit 140 is continuously rotated, the cage 10 is rotated up to about 90 degrees, and the second support wall 19 of the cage 10 comes into contact with a side surface of the rotary member 20, so that rotation of the cage 10 is restrained. In this state, the cage 10 may be inserted between vertebrae. A guide slot 123 may be formed in the push bar 120, and a guide protrusion 113 moving along the guide slot 123 may be formed in the body 110. Further, a structure of the guide slot 123 and the guide protrusion 113 functions to guide linear movement of the push bar 120 and functions as a stopper that restrains a movement range of the push bar 120 as well.

In a state in which the cage 10 is rotated by about 90 degrees, the locking unit 160 is locked to fix the support bar 130 to the second position of the body 110, so that the cage 10 may be inserted while being rotated by 90 degrees. When the cage 10 is completely inserted, the user separates the inner rod 150 from the rotary member 120 of the cage 10 by rotating the rotation knob 152 in a direction that is opposite to a rotation direction when the rotation knob 152 is fastened, and withdraws the apparatus for inserting a cage from a surgical position, that is, the vertebral body 1.

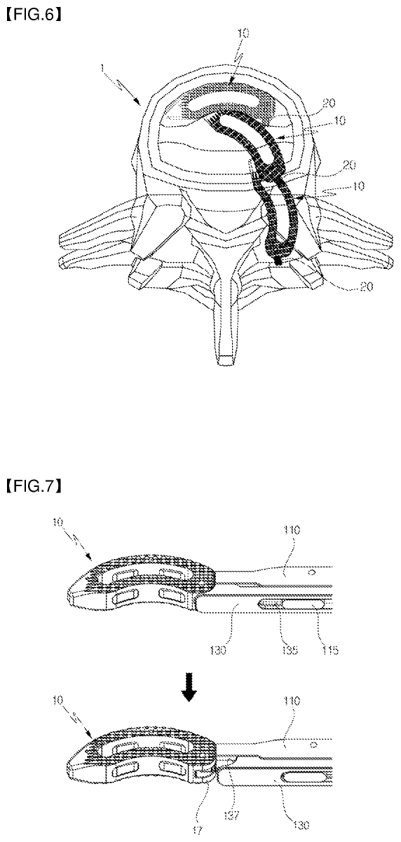

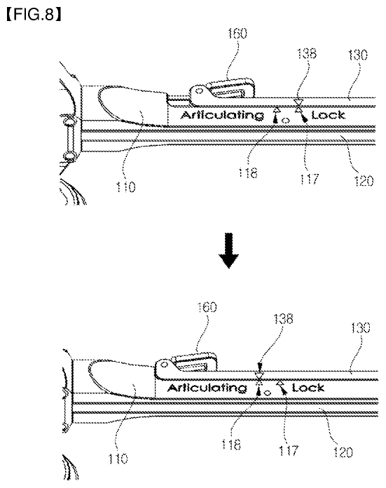

FIGS. 7 and 8 are views illustrating a moving position of a support bar and a state of a locking unit according thereto.

Referring to FIG. 7, a curved surface supporting part 137 supporting a curved surface 17 formed at a tip end of the cage 10 may be formed at a tip end of the support bar 130, and the curved surface supporting part 137, which has a shape corresponding to the curved surface 17, surrounds and supports the curved surface 17. Through such a structure, the cage 10 may be firmly supported when the cage is initially inserted.

A moving slot 135 may be formed through a side surface of the support bar 130 and a guide 115 moving along the moving slot 135 may be provided in the body 110. Linear movement of the support bar 130 is guided by the guide structures 115 and 135, and a final movement location of the support bar 130 is restrained by the guide structures 115 and 135 as well.

Referring to FIG. 8, signs 117, 118 and 138 indicating a rotation state of the cage 10 may be displayed in the body 110 and the support bar 130. According to the present embodiment, the sign 138 having a shape of an arrow is displayed in the support bar 130, the sign 117 having a shape of an arrow, which corresponds to a “Lock” state, and the sign 118 having a shape of an arrow, which corresponds to an “Articulating” state, are displayed in the body 110. Here, the “Lock” state corresponds to the above-described first position, and the “Articulating” state corresponds to the second position. Accordingly, in a state in which the support bar 130 is locked at the first position of the body 110, the sign 138 of the support bar 130 points the sign 117 corresponding to the “Lock” state of the body 110, and in a state in which the support bar 130 is located at the second position of the body 110, the sign 138 of the support bar 130 points the sign 118 corresponding to the “Articulating” state of the body 110. According to such a configuration, a state of the cage 10, that is, whether the cage 10 is locked or is rotated can be easily identified only through identifying the signs without directly locking at the state of the cage 10.

Meanwhile, referring to FIGS. 1 and 2, handles 170 allowing the user to perform surgery while the user holds the handles 170 may be installed in the body 110. According to the present embodiment, the handles 170 may be detachably coupled to the body 110, and to this end, handle coupling parts 180 detachably coupling the handles 170 and the body 110 to each other may be provided between the handles 170 and the body 110.

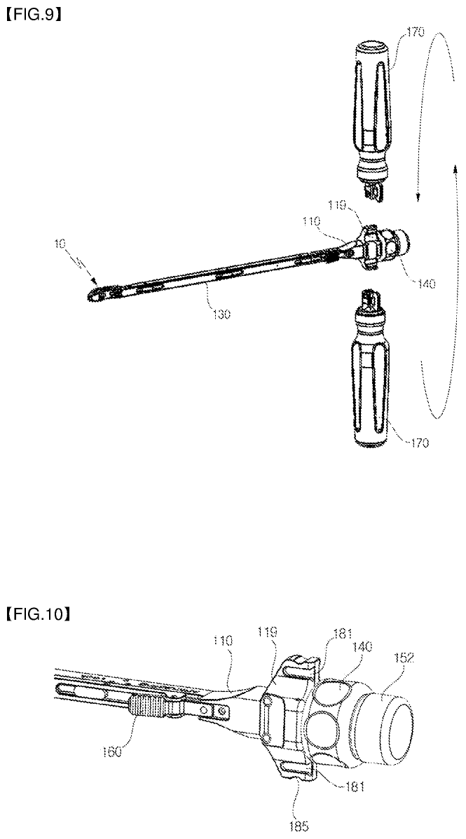

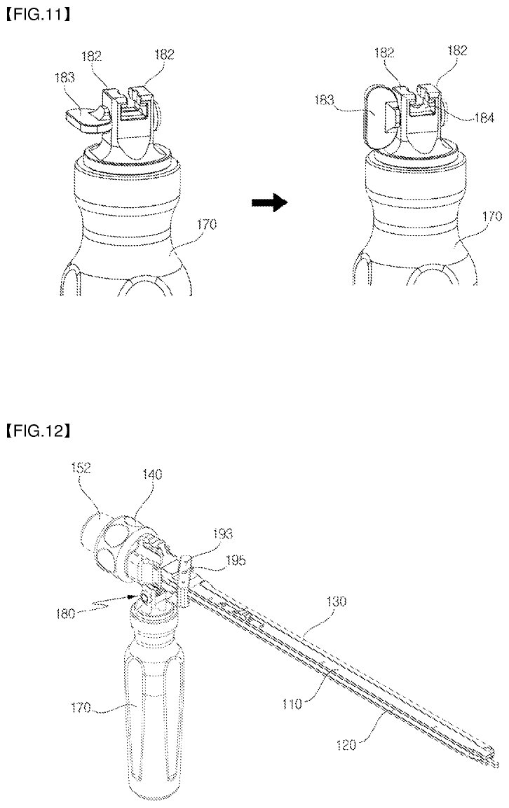

FIG. 9 is a view illustrating an installation form of handles attached to the apparatus for inserting a cage between vertebrae according to the inventive concept, FIGS. 10 and 11 are views illustrating handle coupling parts for coupling of the handles.

Referring to FIG. 9, structures that may couple the handles 170 may be provided at an upper portion and a lower portion of the body 110. Accordingly, there is an advantage in that because the apparatus for inserting a cage may be used while being turned over according to positions to which the handles 170 are coupled, both a right hander and a left hander may install and use the handles 170 in a convenient direction.

Referring to FIGS. 10 and 11, each handle coupling part 180 may include a pair of insertion slots 181 formed in the body 110 and a pair of protrusion inserting parts 182 formed at ends of the handles 170.

Support parts 119 on which the insertion slots 181 are formed may be provided in the body 110. The support parts 119 may be formed at the rear end of the body 110, and may be used as a space in which the above-described power transmission unit is accommodated. The insertion slots 181 may be formed on opposite surfaces of the support parts 119. The above-described support part 119 and the insertion slots 181 may be formed to be vertically symmetric to each other to couple the handles 170 to the upper and lower portions of the body 110.

The pair of protrusion inserting parts 182 are formed at an end of the corresponding handle 170 to define an insertion space for the corresponding support part 119. The protrusion inserting parts 182 are inserted into the insertion slots 181 of the support part 119 inserted therebetween.

A fixing unit 183 configured to fix or release the support part 119 may be installed in the protrusion inserting parts 182 to be rotatable. According to the present embodiment, the fixing unit 183 may have a catching part 184 that is caught by a catching groove 185 formed at a lower end of the support 119 and the catching part 184 may be arranged inside the catching groove 185 as the catching part 184 is rotated according to rotation of the fixing unit 183. According to such a configuration, after the protrusion inserting parts 182 are inserted into the insertion slots 181 while the support part 119 is inserted into a space between the pair of protrusion inserting parts 182, the catching part 184 is located inside the catching groove 185 by rotating the fixing unit 183, so that the handle 170 may be fixed to the body 110. Further, the handle may be separated when the above-described process is performed in an inverse order.

The above-described handle coupling parts 180 are illustrative, and may be implemented in various modified configurations as long as the configurations may detachably couple the handles 170 to the body 110.

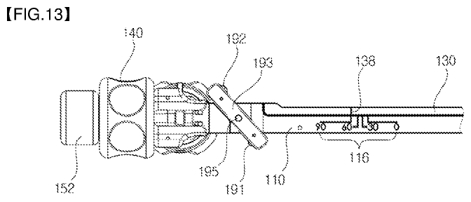

FIG. 12 is a perspective view illustrating an apparatus for inserting a cage between vertebrae according to another embodiment of the inventive concept; and, FIG. 13 is a plan view illustrating the apparatus for inserting a cage between vertebrae, which is illustrated in FIG. 12.

The apparatus for inserting a cage between vertebrae according to the present embodiment is basically the same as that according to the prior embodiment, and is different from that according to the prior embodiment in that some configurations of the power transmission unit is installed outside the body 110 and in terms of expression of the signs indicating the rotation state of the cage 10. Descriptions of configurations which are the same as that according to the prior embodiment will be replaced with the above descriptions.

According to the present embodiment, the power transmission unit includes first and second connectors 191 and 192 extending from side surfaces of the push bar 120 and the support bar 130, and a rotary body 193, opposite ends of which are connected to the first and second connectors 191 and 192 to be rotatable. The rotary body 193 is installed outside the body 110 to be rotated according to the rotation of the adjustment unit 140 (rotary handle). The rotary shaft 195 is fixed to the rotary body 193 vertically extending and a power transmission structure (for example, a bevel gear or the like) may be embedded in the body 110 between the rotary shaft 195 and the adjustment unit 140.

The rotary shaft 195 is rotated according to the rotation of the adjustment unit 140 so that the rotary body 193 may be pivoted, the push bar 120 and the support bar 130 connected to the rotary body 193 to be rotatable may be moved in directions that are opposite to each other. FIGS. 12 and 13 illustrate a state in which the rotary body 193 is maximally rotated in a counterclockwise direction so that the push bar is maximally moved forwards and the support bar 130 is maximally moved rearwards.

For reference, a linear movement structure of the push bar 120 and the support bar 130 through rotation of the rotary body 193 may be used as the power transmission structure or the user may directly manipulate the rotation of the rotary body 193 to adjust the locations of the push bar 120 and the support bar 130. In this case, the rotary body 193 functions as the adjustment unit 140 according to the prior embodiment, and in this case, the rotary handle may not be installed.

Meanwhile, according to the present embodiment, the signs 116 and 138 indicating the rotation state of the cage 10 are displayed to indicate a rotation angle of the cage 10. According to the present embodiment, the sign 116 configured to display the rotation angle of the cage 10 in stages is displayed as “0°, 30°, 60°, 90°” Accordingly, there is an advantage in that a posture according to the rotation angle of the cage 10 may be identified more easily than the state in which only two states including the “Lock” state and the “Articulating” state are indicated according to the prior embodiment. As above, the signs indicating the rotation state of the cage 10 may display the state or the rotation angle of the cage 10 in various forms such as a character, a symbol and a number.

According to the inventive concept, a posture of a cage may be stably maintained in a process of inserting a cage through a support structure and a locking structure of a push bar and a support bar.

Further, according to the inventive concept, when the cage is rotated, the posture of the cage may be precisely adjusted through a structure that adjusts movement amounts of the push bar and the support bar by adjusting the adjustment unit.

Further, according to an articulated rotary motion structure using a moving mechanism of the push bar and the support bar, a rotation angle of the cage may be increased to 0 to 90 degrees, which is a range larger than that of the conventional case.

Further, components of the apparatus for inserting a cage are configured to be detachable, so that the components may be simply separated and coupled without an additional tool after surgery, and handles are configured to be vertically detachable, so that both a right hander and a left hander may use the apparatus while attaching the handles in a convenient direction.

The above-described apparatus for inserting a cage between vertebrae is not limited to the configurations and methods according to the above embodiments, but the entireties or portions of the above embodiments may be selectively combined so that various modifications may be achieved.

1. An apparatus for inserting a cage between vertebrae, the apparatus comprising:
a body accommodating an inner rod coupled to the cage, which is to be inserted between vertebrae;
a push bar installed in the body to be slid and configured to push one side of the cage as the push bar is moved forwards from the body;
a support bar supporting an opposite side of the cage and installed in the body to be slid to move rearwards when the push bar is moved forwards; and
an adjustment unit installed in the body to be manipulated and configured to adjust movements of the push bar and the support bar such that a rotation amount of the cage is adjusted according to manipulation by a user.

2. The apparatus of claim 1, wherein a curved surface supporting part supporting a curved part of the cage is formed at a tip end of the support bar.

3. The apparatus of claim 1, wherein the adjustment unit comprises:
a rotary handle installed in the body to be rotatable.

4. The apparatus of claim 1, wherein a power transmission unit configured to move the support bar and the push bar in opposite directions as the adjustment unit is rotated is installed inside the body.

5. The apparatus of claim 4, wherein the power transmission unit comprises:
first and second connectors extending from side surfaces of the push bar and the support bar, respectively; and
a rotary body, opposite ends of which are connected to the first and second connectors to be rotatable, and which formed in the body to be pivoted as the adjustment unit is rotated.

6. The apparatus of claim 1, further comprising a locking unit configured to lock and unlock the support bar at a first position of the body when the cage is initially inserted between vertebrae.

7. The apparatus of claim 6, wherein the locking unit comprises:
a manipulation lever installed in the support bar to be rotatable; and
a first coupling part provided in the body and detachably coupled to the manipulation lever.

8. The apparatus of claim 7, wherein the locking unit further comprises:
a second coupling part detachably coupled to the manipulation lever to lock and unlock the support bar at a second position of the body when the cage is rotated.

9. The apparatus of claim 6, wherein signs indicating a rotation state of the cage are displayed on the body and the support bar.

10. The apparatus of claim 9, wherein the signs comprise:
at least one of a character, a symbol and a number indicating the rotation state or an angle of the cage.

11. The apparatus of claim 1, further comprising:
handles coupled to the body; and
handle coupling parts configured to detachably couple the handles to the body.

12. The apparatus of claim 11, wherein each of the handle coupling parts comprises:
insertion slots formed on opposite surfaces of a support part formed in the body; and
a pair of protrusion inserting parts formed at an end of the corresponding handle to define an insertion space of the support part and inserted into the insertion slots.

13. The apparatus of claim 12, wherein the support parts and the insertion slots are symmetrically formed at upper and lower portions of the body such that the handles are coupled to the upper and lower portions of the body.

14. The apparatus of claim 12, wherein the handle coupling part further comprises:
a fixing unit installed in the protrusion inserting parts to be rotatable to lock or unlock the support part.
